(19) United States
(12) Patent Application Publication
(10) Pub. No.: US 2006/0110804 A1
(43) Pub. Date: May 25, 2006

(54) Method of treating an organ for transplantation

(76) Inventor: Makoto Suematsu, Tokyo (JP)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/996,504

(22) Filed: Nov. 24, 2004

(51) Int. Cl. C12P 19/08

(52) U.S. Cl. 435/103

(57) ABSTRACT

The present invention provides a method of treating an organ for transplantation, in which an organ graft is cold-preserved with an organ preservation solution for a given period of time and reperfused with a solution containing an intracellular accessibility, lipophilic small-molecular antioxidant (e.g., bilirubin) to wash the organ graft before being transplanted. Livers, hearts, kidneys, pancreases or lungs are suitable as organs for transplantation.

### FIELD OF THE INVENTION

The present invention relates to a method of treating an organ for transplantation, a treatment agent for organ graft rinse used therein, a method of organ transplantation, and a method of organ preservation.

### BACKGROUND OF THE INVENTION

Organ transplantation, particularly cadaveric organ transplantation, involves the following procedures: washing of donor organ grafts, perfusion of the organ grafts with preservation solution, cold preservation of the organ grafts and reperfusion with warm irrigation solution to rinse off the remaining preservation solution before transplantation, and transplantation into recipients. However, cold preservation and reperfusion of organ grafts are believed as unavoidable and harmful processes which interfere with graft function recovery after transplantation. In both living donor liver transplantation and cadaveric liver transplantation, prevention of cold ischemia/reperfusion (CI/R)-induced acute biliary/liver dysfunction is an important problem to be solved in achieving further improvements in post-transplantation results (see, e.g., Documents 1 and 2). Early graft dysfunction is known as an example of serious form of CI/R-induced biliary/liver dysfunction (see, e.g., Document 3). One possible cause may be that inflammation-inducible mediators produced upon CI/R, such as oxygen free radicals and inflammatory cytokines, will cause injury to liver cells and/or liver non-parenchymal cells; and there are many series of evidence reports suggesting this possibility (e.g., Document 4). On the other hand, it has been experimentally shown that cold ischemic preconditioning of donor livers allows liver grafts to attain resistance to reperfusion injury. In relation to such cold ischemic preconditioning of grafts, the following are believed to be effective: inactivation or depletion of astrocytes (see, e.g., Documents 5 and 6), induction of heat shock proteins (see, e.g., Documents 7 and 8), short-term ischemia prior to cold preservation (see, e.g., Document 9), etc.

Moreover, recent studies have reported that gene transfer-induced or enzyme inducer-induced overexpression of heme oxygenase 1 (HO-1) is effective for improving post-transplantation survival of liver grafts in animals (see, e.g., Documents 10 and 11). Such an enzyme is an isozyme of heme oxygenase, which cleaves protoheme IX (protoporphyrin IX) at the α-methylene bridge into free divalent iron, carbon monoxide (CO) and biliverdin-IXα. Biliverdin-IXα is a substrate that is further cleaved with biliverdin reductase to produce bilirubin-IXα (see, e.g., Documents 12 and 13). These substances produced from protoporphyrin IX are biologically active. More specifically, divalent iron acts as an endogenous inducer of ferritin and hence facilitates the storage of free iron in cells (see, e.g., Documents 14 and 15). CO is a substance necessary for maintaining the blood flow and patency of sinusoidal capillaries under normal and disease conditions (e.g., Document 16 to 18). Biliverdin and bilirubin have been reported to have the function of eliminating reactive oxygen species in vitro (see, e.g., Document 19). Likewise, there is a report indicating that pretreatment of genetically obese Zucker rats with a HO-1 inducer such as metalloprotoporphyrin significantly improved graft viability; HO-1 gene transfer supports long-term graft viability in animals after liver and cardiac transplantation.

The following are also known: a method for measuring HO-1 protein expression by Western blot analysis (see, e.g., Document 20); a method for measuring the concentration of bilirubin-IXα in bile samples (see, e.g., Documents 21 and 22); a method for measuring the total amounts of bile salts and phospholipids in bile samples (see, e.g., Document 23); a method for measuring the level of lactate dehydrogenase (LDH) in samples (see, e.g., Document 24); a method for liver transplantation in rats (see, e.g., Document 25); a human plasma protein fraction (PPF)-containing irrigation solution for use in recirculation after cold preservation (see, e.g., Document 26); a method for immunohistological detection of bilirubin using the anti-biliverdin-IXα monoclonal antibody 24G7 (see, e.g., Document 27); and the level of bilirubin secretion (see, e.g., Document 28) and the concentration of plasma bilirubin (see, e.g., Document 29) in liver grafts after 16-hour cold preservation and 30 minute reperfusion. In addition, reactive oxygen (e.g., oxygen free radicals) generated in vivo by, e.g., neutrophil phagocytosis of bacteria having invaded into the body is responsible for inflammation, tissue damage, etc. To eliminate such reactive oxygen, water-soluble drugs are difficult to reach target sites at effective concentrations because they will be metabolized and decomposed in the digestive tract and liver when administered orally, while they will be transferred to the blood, but not incorporated into cells when administered parenterally. For this reason, a lymptropic composition for scavenging free radicals has been reported, which is designed for percutaneous administration of essential oil components such as eucalyptus oil and thyme oil to enhance the lymphatic transfer of drugs, that is, which comprises at least one essential oil component containing a number of lipophilic small-molecular compounds (see, e.g., Document 30).

### SUMMARY OF THE INVENTION

Because of its protective effect on liver grafts, preconditioning with HO-1 can be expected to expand the supply of usable donor livers. However, in spite of these experimental data supporting the protective effect of HO-1, apart from recent studies showing the beneficial effect of CO perfusion on graft survival ex vivo, it is still unknown what role the reaction products play in ameliorating graft injury. In human liver transplantation, solutions containing high concentrations of plasma proteins have been conventionally used as rinse solutions for grafts. Rinsing with these rinse solutions can prevent cell injury, but fails to improve the bile secretory function and is also associated with problems of virus infection and costs because the solutions are plasma formulations. For this reason, there has been a strong demand for a safer and simpler method of treating organs for transplantation, which is intended for transplantation protection against post-transplantation reperfusion injury (e.g., post-transplantation organ dysfunction) in organ transplantation and which is capable of preventing early graft dysfunction.

The object of the present invention is to elucidate the mechanism causing post-transplantation reperfusion injury (e.g., post-transplantation organ dysfunction) in organ transplantation and to provide a safer and simpler method of treating an organ for transplantation, which is intended for improving the bile secretory function to prevent early graft dysfunction due to post-transplantation reperfusion injury and which eliminates the need for treatment of virus infection. The present invention also aims to provide a treatment agent for organ graft rinse used in the above method, a method of organ transplantation, and a method of organ preservation.

As a result of extensive and intensive efforts made to elucidate the mechanism of action whereby HO-1 preconditioning has an effect on liver graft dysfunction induced by cold ischemia/reperfusion (CI/R) in vivo. The inventors have elucidated that liver damage due to cold ischemic preservation is liver parenchymal cell damage although it has been believed to be sinusoidal endothelial cell damage. They have also confirmed that bilirubin-IXα, a bile pigment, is a more important factor than CO for amelioration of CI/R-induced biliary/liver dysfunction. They have found that when organs are transplanted after being reperfused with irrigation solution (e.g., lactated Ringer's solution) simply supplemented with bilirubin at a concentration around 5 to 10 μmol/L, the same organ protective effect as provided by HO-1 preconditioning can be achieved even in the absence of HO-1 preconditioning, thus significantly preventing CI/R-induced dysfunction in vivo and remarkably improving liver tissue damage and bile secretion at 24 hours after transplantation. This finding led to the completion of the present invention.

Namely, the present invention is as follows.

(1) A method of treating an organ for transplantation, which comprises perfusing an organ graft with an organ preservation solution, preserving the organ graft under cold conditions for a given period of time, and reperfusing the organ graft with an antioxidant-containing solution, which is to be incorporated into cells, to wash the organ graft before being transplanted.

(2) The method of treating an organ for transplantation according to (1) above, wherein the antioxidant-containing solution to be incorporated into cells is a bilirubin-containing solution.

(3) The method of treating an organ for transplantation according to (2) above, wherein the bilirubin-containing solution is a 5 to 10 μmol/L bilirubin-containing solution.

(4) The method of treating an organ for transplantation according to (2) or (3) above, wherein the bilirubin-containing solution is reperfused for 3 to 10 minutes to wash the organ graft.

(5) The method of treating an organ for transplantation according to any one of (1) to

(4) above, wherein the organ is liver, heart, kidney, pancreas or lung.

(6) A treatment agent for organ graft rinse used in reperfusing an organ graft, which has been perfused with an organ preservation solution and then cold-preserved for a given period of time, to wash the organ graft before being transplanted, wherein said treatment agent for organ graft rinse comprises, as an active ingredient, an antioxidant-containing solution to be incorporated into cells.

(7) The treatment agent for organ graft rinse according to (6) above, wherein the antioxidant-containing solution to be incorporated into cells is a bilirubin-containing solution.

(8) The treatment agent for organ graft rinse according to (7) above, wherein the bilirubin-containing solution is a 5 to 10 μmol/L bilirubin-containing solution.

(9) The treatment agent for organ graft rinse according to any one of (6) to (8) above, wherein the organ is liver, heart, kidney, pancreas or lung.

(10) A method of organ transplantation, which comprises perfusing an organ graft with an organ preservation solution, preserving the organ graft under cold conditions for a given period of time, reperfusing the organ graft with an antioxidant-containing solution, which is to be incorporated into cells, to wash the organ graft before being transplanted, and transplanting the organ graft.

(11) The method of organ transplantation according to (10) above, wherein the organ transplantation is isograft transplantation.

(12) The method of organ transplantation according to (10) or (11) above, wherein the antioxidant-containing solution to be incorporated into cells is a bilirubin-containing solution.

(13) The method of organ transplantation according to (12) above, wherein the bilirubin-containing solution is a 5 to 10 μmol/L bilirubin-containing solution.

(14) The method of organ transplantation according to (12) or (13) above, wherein the bilirubin-containing solution is reperfused for 3 to 10 minutes to wash the organ graft.

(15) The method of organ transplantation according to any one of (10) to (14) above, wherein the organ is liver, heart, kidney, pancreas or lung.

(16) A method of organ preservation, which comprises perfusing an organ with an organ preservation solution, preserving the organ under cold conditions for a given period of time, and reperfusing the organ with an antioxidant-containing solution, which is to be incorporated into cells, to wash the organ before being used.

(17) The method of organ preservation according to (16) above, wherein the antioxidant-containing solution to be incorporated into cells is a bilirubin-containing solution.

(18) The method of organ preservation according to (17) above, wherein the bilirubin-containing solution is a 5 to 10 μmol/L bilirubin-containing solution.

(19) The method of organ preservation according to (17) or (18) above, wherein the bilirubin-containing solution is reperfused for 3 to 10 minutes to wash the organ.

(20) The method of organ preservation according to any one of (16) to (19) above, wherein the organ is liver, heart, kidney, pancreas or lung.

### BRIEF DESCRIPTION OF THE DRAWINGS

FIG. 1 shows the results of the effects of hemin treatment on ex vivo reperfused liver grafts.

(A) HO-1 protein expression determined by Western blotting

(B) Venous CO fluxes

FIG. 2 shows hemin treatment-induced improvement of liver functions in ex vivo reperfused liver grafts.

(A) Time course of bile output

(B) Bilirubin levels in bile after 30 minute reperfusion

FIG. 3 shows the effects of bilirubin on the recovery of HO-1 inhibitor-induced dysfunction in ex vivo reperfused liver grafts.

(A) Bilirubin-induced recovery of bile production under inhibition of hemin treatment effects

(B) Bilirubin-induced recovery of bile salt production under inhibition of hemin treatment effects

(C) Bilirubin-induced recovery of phospholipid production under inhibition of hemin treatment effects

FIG. 4 shows the dose-dependent effects of bilirubin on the recovery of HO-1 inhibitor-induced dysfunction in ex vivo reperfused liver grafts.

(A) Bilirubin-induced recovery of bile production under inhibition of hemin treatment effects

(B) Bilirubin-induced recovery of bile salt production under inhibition of hemin treatment effects

(C) Bilirubin-induced recovery of phospholipid production under inhibition of hemin treatment effects

FIG. 5 shows the dose-dependent effects of bilirubin on the recovery of HO-1 inhibitor-induced liver cell damage (venous LDH concentration) in ex vivo reperfused liver grafts.

FIG. 6 shows the recovery of liver dysfunction and liver cell damage in liver grafts at various time points of ex vivo bilirubin reperfusion.

(A) Time course of bile production at various time points of bilirubin reperfusion

(B) Bile production levels at various time points of bilirubin reperfusion

(C) Venous LDH concentrations at various time points of bilirubin reperfusion

FIG. 7 shows that bilirubin is responsible for in vivo improvement of liver functions and in vivo recovery of liver cell damage after liver graft transplantation.

(A) Bile output levels at 30 minutes after initiation of reperfusion

(B) Bile output levels at 24 hours after initiation of reperfusion

(C) Serum AST levels at 24 hours after initiation of reperfusion

(D) Serum ALT levels at 24 hours after initiation of reperfusion

(E) Serum LDH levels at 24 hours after initiation of reperfusion

FIG. 8 shows the results of immunohistochemical analysis illustrating the in vivo accessibility of bilirubin to parenchymal cells in the transplanted tissue after liver graft transplantation.

(A) Graft perfused with bilirubin-free lactated Ringer's solution

(B) Negative control in the absence of anti-bilirubin antibody

(C) Graft perfused with bilirubin-containing lactated Ringer's solution

(D) Graft perfused with bilirubin-containing lactated Ringer's solution supplemented with PPF

### DETAILED DESCRIPTION OF THE INVENTION

The method of treating an organ for transplantation according to the present invention is not limited in any way, as long as it involves perfusing an organ graft with an organ preservation solution, preserving the organ graft under cold conditions for a given period of time, and reperfusing the organ graft with an antioxidant-containing solution, which is to be incorporated into cells, to wash (rinse) the organ graft before being transplanted. The treatment agent for organ graft rinse according to the present invention is not limited in any way, as long as it is used in reperfusing an organ graft, which has been perfused with an organ preservation solution and then cold-preserved for a given period of time, to wash the organ graft before being transplanted, and also as long as it comprises, as an active ingredient, an antioxidant-containing solution to be incorporated into cells. The method of organ transplantation according to the present invention is not limited in any way, as long as it involves perfusing an organ graft with an organ preservation solution, preserving the organ graft under cold conditions for a given period of time, reperfusing the organ graft with an antioxidant-containing solution, which is to be incorporated into cells, to wash the organ graft before being transplanted, and transplanting the organ graft. The method of organ preservation according to the present invention is not limited in any way, as long as it involves perfusing an organ with an organ preservation solution, preserving the organ under cold conditions for a given period of time, and reperfusing the organ with an antioxidant-containing solution, which is to be incorporated into cells, to wash the organ before being used. The organ for transplantation used in these embodiments of the present invention may be either a living donor organ or a viable cadaver organ. Types of organs include livers, hearts, kidneys, lungs and pancreases, with livers being preferred. In particular, viable cadaver livers can be preferably exemplified. The whole or part of such an organ may be used as a graft.

Although any organ preservation solution, including known ones, can be used for the above purpose, the solution may be selected and used as most appropriate for the type of organ to be preserved, etc. For example, in a case where the organ for transplantation is a cadaver liver, the organ preservation solution may be exemplified by University of Wisconsin solution (UW solution) and Euro-Collins solution, which are lactated Ringer's solutions containing inhibitors of reactive oxygen-producing enzymes and for which preservative effects have been clinically reported to continue for an average of 24 hours in kidneys, for an average of 17 hours in livers/pancreases, and for an average of 4 hours in hearts, as well as an organ preservation solution containing lecithin-modified superoxide dismutase (JP 2002-060301 A), an organ preservation solution containing hyaluronates (JP 11-246301 A), an organ preservation solution containing 2% to 3.5% hydroxyethyl starch having an average molecular weight of 500,000 to 650,000 (JP 09-328401 A), a transplant organ preservation solution containing diisopropyl 1,3-dithiol-2-ylidene malonate as an active ingredient (JP 2001-335401 A), etc. These organ preservation solutions are usually used by perfusing them through donor organ grafts after flushing. The organ grafts perfused with these organ preservation solutions are then cold-preserved for a given period of time. Cold preservation is preferably performed at preservation temperatures optimum for the individual organ grafts, usually at a temperature around 4° C. Preservation may be continued for any period of time within a range permitted for each organ graft, preferably within 16 hours for liver and pancreas grafts, within 24 hours for kidney grafts, and within 4 hours for hearts, although depending on the type of preservation solution.

In the present invention, rinsing of organ grafts after cold preservation and before transplantation, preferably immediately before transplantation, is not limited in any way, as long as it involves reperfusing organ grafts with an antioxidant-containing solution to be incorporated into liver cells. Any solution may be used as an antioxidant-containing solution to be incorporated into cells, as long as it contains a fat-soluble antioxidant, preferably a lipophilic small-molecular antioxidant, which is incorporated into cells when perfused through organ tissues, or an antioxidant capable of serving as a transporter-specific substrate which is incorporated into cells via a transporter, etc. As used herein, the term “antioxidant” refers to a substance capable of preventing oxidant-induced lipid peroxidation at low concentrations, and also includes substances that react with and thereby scavenge reactive oxygen (e.g., superoxide anions, hydroxy radicals, hydrogen peroxide) generated in vivo. The above fat-soluble antioxidant is incorporated into cells and those of lower molecular weight will be more readily incorporated into cells.

Examples of such an intracellular accessibility, lipophilic small-molecular antioxidant include bilirubin, biliverdin, vitamin E, β-carotene and lycopene, as well as naturally-occurring compounds contained in various plant essential oils, such as phenol compounds contained in thyme oil, Lamiaceae essential oil, eucalyptus oil and the like, sulfur-containing compounds contained in garlic oil, shallot oil, scallion oil and the like, and terpene compounds contained in lemon oil, citrus oil, lavender oil and the like. Specific examples of antioxidants contained in these plant essential oils include thymol, carvacrol, diallylsulfide, diallyldisulfide, allicin, diallyltrisulfide, d- or l-camphene, β-eudesmol, dipentene (dl-limonene), d-linalol, linalyl acetate, limonene, citral, and terpineol. In particular, when the organ to be transplanted is the liver, bilirubin can be given as a preferred example of such a lipophilic small-molecular antioxidant.

The above antioxidant capable of serving as a transporter-specific substrate which is incorporated into cells via a transporter may be exemplified by uric acid transported via the organic anion transporter OAT1 (J. Biol. Chem., 272, 18526-18529, 1997) expressed in the kidney, as well as glutathione transported via the ABC transporter MRP2 (Science, 271, 1126-1128, 1996) expressed in the liver, etc.

These antioxidants incorporated into cells may be used alone or in combination. Moreover, the above antioxidant-containing solution to be incorporated into cells may further comprise, in addition to an antioxidant, an inorganic compound (e.g., sodium chloride, potassium chloride, calcium chloride) and other ingredients (e.g., lactate) contained in commonly used organ preservation solutions.

Bilirubin used for the above purpose may be either synthesized or obtained as a final product from hemoglobin. Bilirubin is produced from a hemoglobin component, porphyrin-iron(II) complex salt (heme), which is cleaved at the α-methine position by the action of heme oxygenase into a linear tetrapyrrole derivative (biliverdin) and further converted by the action of biliverdin reductase into a hemoglobin final product. The resulting bilirubin-IXα is unconjugated and lipophilic. The content of bilirubin in a lipophilic small-molecular antioxidant-containing solution may be 1 to 20 lmol/L, preferably 5 to 10 μmol/L.

In the present invention, reperfusion for organ graft washing with an antioxidant-containing solution to be incorporated into cells allows the antioxidant (e.g., a lipophilic small-molecular antioxidant) to penetrate into cells in the grafts, thus enabling efficient elimination of intracellular reactive oxygen (e.g., oxygen free radicals) generated during reperfusion subsequent to cold ischemic preservation. In particular, bilirubin highly penetrates into liver parenchymal cells and thus a bilirubin-containing solution is preferred for use. In a case where an organ graft is the liver, reperfusion for organ graft washing is preferably accomplished by slowly injecting a lipophilic small-molecular antioxidant-containing solution from the portal opening to perfuse and rinse the liver because it is possible to promote cell penetration of the antioxidant to be incorporated into cells. Although the time required for reperfusion will vary depending on the types of organ and antioxidant to be incorporated into cells, it may be selected as most appropriate for each combination between organ and antioxidant. For example, in a case where a bilirubin-containing solution is reperfused to wash liver grafts, reperfusion is preferably performed for 3 to 10 minutes. Within this time period, the right amount of bilirubin will penetrate into cells through perfusion/rinsing and can efficiently eliminate reactive oxygen in the cells.

The method of treating an organ for transplantation according to the present invention is useful as treatment of transplant organs on the occasion of organ transplantation. The treatment agent for organ graft rinse according to the present invention can be advantageously used for treatment of transplant organs on the occasion of organ transplantation. The method of organ transplantation according to the present invention is advantageously used for isograft transplantation, etc. Likewise, a variety of animal (e.g., rat) organs preserved by the method of organ preservation according to the present invention are advantageously used in various experiments such as those for transplantation.

### EXAMPLE

The present invention will be further described in more detail in the following Examples, which are not intended to limit the technical scope of the invention. In the following experiments, all experimental procedures used in animal housing and handling were approved by the Keio University, School of Medicine, Laboratory Animal Care and Use Committee. The statistical analysis of each experimental data between groups was performed by one-way analysis of variance and the Fisher's multiple comparison test. Statistical differences in data between free bilirubin group and albumin-conjugated bilirubin group were examined by non-parametric analysis using the Mann-Whitney's U test. The results were expressed as mean±standard error, and P<0.05 was considered statistically significant.

[Ex Vivo Experiments]

### Example 1

### Preparation and HO-1 Preconditioning of Liver Grafts

Male Wistar rats of 190-230 g were allowed to take pellet chow and water without any restriction and then fasted for 6 hours. Subsequently, the rats were intraperitoneally injected with 40 μmol/kg hemin, a strong HO-1 inducer, and then fasted for 18 hours. Hemin treatment was performed according to a reported method (see Document 17), indicating that hemin treatment caused significant increases in hepatic venous CO fluxes and bilirubin-IXα secretion into bile, with maximal induction of HO-1 protein. Based on this result, the time period mentioned above was selected as an optimum operation to examine the effect of HO-1 induction against graft dysfunction following cold ischemia. As a hemin-untreated control, rats injected with physiological saline were fasted for 24 hours and then used for the experiment.

The rats injected with hemin or physiological saline were anesthetized by intraperitoneal injection of pentobarbital sodium (40 mg/kg body weight) and a PE-10 catheter for bile sample collection was inserted into the common bile duct of each rat. A 16-gauge catheter was inserted into the portal vein and 20 ml lactated Ringer's solution kept at 4° C. was passed through the catheter. Subsequently, rat livers were perfused with 20 ml University of Wisconsin solution (UW solution) of 4° C., excised and then preserved in the same solution at 4° C. for 16 hours. Since acute oxidative stress would eventually occur, as judged by intracellular hydroperoxide production which occurred concurrently with a reduction of bile formation, a time period of 16 hours was selected for preservation purposes. After cold preservation for 16 hours, each liver graft was reperfused via the portal vein with 95% oxygen-5% carbon dioxide-saturated Krebs-Henseleit buffer (pH 7.4, 37° C.) at 4 ml/min/g liver in the presence of 30 μmol/L sodium taurocholate (see Document 4).

### Example 2

### HO-1 Protein in Hemin Treatment of Grafts

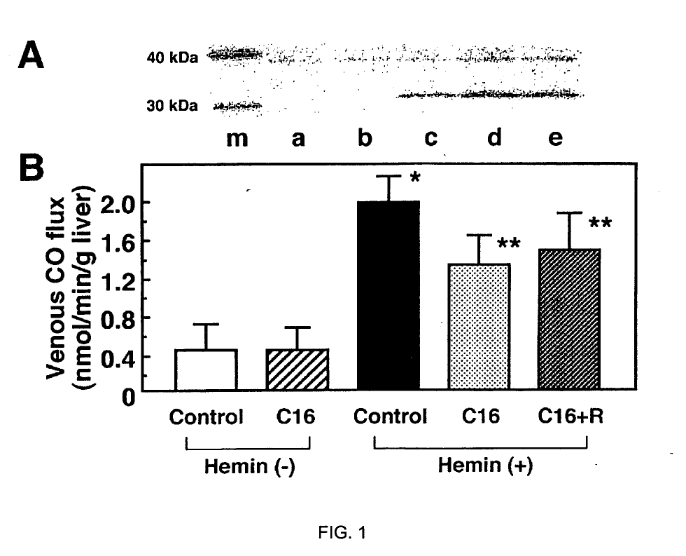

The grafts prepared in Example 1 were measured for HO-1 protein expression by Western blot analysis, as described in an earlier document (see Document 20). CO fluxes in hepatic venous flow were also measured with a spectrophotometer according to a previously reported method of the inventors (see Document 16). The results obtained are shown in FIGS. 1(A) and (B). FIG. 1(A) shows HO-1 protein expression in each graft, as measured by Western blot analysis using the anti-rat HO-1 monoclonal antibody GTS-1. Lane m represents molecular markers (30 kDa and 40 kDa), Lanes a and b represent hemin-uninjected (hemin-untreated) grafts receiving no cold preservation (Lane a) and 16 hour-cold preservation (Lane b), respectively. Lanes c, d and e represent hemin-injected (hemin-treated) grafts receiving no cold preservation (Lane c), 16-hour cold preservation (Lane d), and 16-hour cold preservation and subsequent 30 minute reperfusion (Lane e), respectively. FIG. 1(B) shows CO levels in hepatic venous perfusate from each graft. The figure shows the results of hemin-untreated grafts receiving no cold preservation (Control) and 16-hour cold preservation (C16), as well as the results of hemin-treated grafts receiving no cold preservation (Control), 16-hour cold preservation (C16), and 16-hour cold preservation and subsequent 30 minute reperfusion (C16+R).

As shown in FIG. 1, the hemin-untreated grafts receiving 16-hour cold preservation (Lane b, C16) showed no detectable HO-1 expression and no increase in CO output. The hemin-treated grafts showed significant increases in both HO-1 expression and venous CO fluxes when compared to the hemin-untreated grafts. The grafts receiving cold preservation (Lane d, C16) and the grafts receiving cold preservation and subsequent 30 minute reperfusion (Lane e, C16+R) showed a slight increase in HO-1 expression levels over the grafts receiving no cold preservation (Lane c, Control). On the other hand, the grafts receiving cold preservation (Lane d, C16) and the grafts receiving cold preservation and subsequent 30 minute reperfusion (Lane e, C16+R) showed a decrease in hepatic venous CO fluxes over the grafts receiving no cold preservation (Lane c, Control), but they showed about a 3-fold increase in this parameter when compared to the hemin-untreated grafts. These results suggested that the hemin-treated liver grafts increased their CO-producing capacity through HO-1 induction.

### Example 3

### Hemin Treatment-Induced Production of Bile and Bilirubin in Grafts

In the hemin-treated liver grafts from Example 1, after 16-hour cold preservation (Hemin/C16), bile samples were collected from the common bile duct every 5 minutes during 40 minute reperfusion. The bile samples collected at 30 minutes after initiation of reperfusion were also measured for their bilirubin-IXα concentration by enzyme-linked immunosorbent assay detection using the monoclonal antibody 24G7 capable of recognizing both conjugated and unconjugated bilirubin fractions, as described in earlier documents (see Documents 21 and 22). The hemin-untreated grafts receiving no cold preservation (Control) and 16-hour cold preservation (C16) were also provided for the same measurement. To examine the role of HO activity in the prevention of reperfusion-induced graft dysfunction, the hemin-treated liver grafts receiving 16-hour cold preservation were reperfused with carbogen-saturated Krebs-Henseleit buffer (pH 7.4, 37° C.) supplemented with 1 μmol/L zinc protoporphyrin-IX (ZnPP, Porphyrin Products, Inc.; a strong HO-1 activity inhibitor) or 1 μmol/L copper protoporphyrin-IX (CuPP, Porphyrin Products Inc.; not inhibit HO activity), followed by measuring the level of bile output from the grafts and the bilirubin concentration in the bile samples collected after 30 minute perfusion.

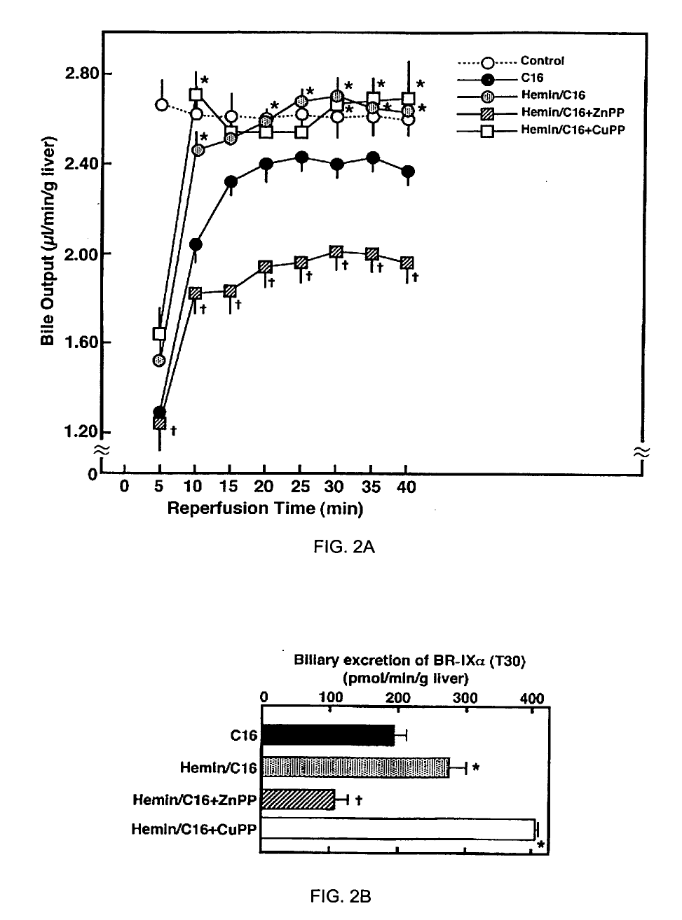

FIG. 2(A) shows the time course of bile output during reperfusion. As shown in FIG. 2(A), in the hemin-untreated groups, bile output from the grafts receiving 16-hour cold preservation (C16) reached a plateau over time, but its level was significantly lower than that of the hemin-untreated grafts receiving no cold preservation (Control). This result supports the observation that liver grafts cold-preserved for 16 hours will develop liver/biliary dysfunction, and it is well consistent with the previous studies of the inventors (see Document 4). In the hemin-treated groups, in contrast, bile output from the grafts receiving 16-hour cold preservation (Hemin/C16) showed a rapid recovery as early as 10 minutes after initiation of reperfusion and was found to reach a higher plateau than that of the hemin-untreated cold-preserved grafts (C16).

Likewise, as shown in FIG. 2(A), the presence of ZnPP reduced bile output during graft reperfusion (Hemin/C16+ZnPP), and the difference in bile output levels compared to the hemin-untreated cold-preserved grafts (C16) was clearly greater than that observed between the hemin-untreated cold-preserved grafts (C16) and the hemin-treated cold-preserved grafts (Hemin/C16). The grafts reperfused using CuPP instead of ZnPP (Hemin/C16+CuPP) showed substantially the same bile output as the hemin-treated cold-preserved grafts (Hemin/C16). This estimated that ZnPP would inhibit not only HO-1 activity, but also the functions of cells in the grafts. This estimation was fully supported by measurement of bilirubin-IXα levels in bile.

FIG. 2(B) shows bilirubin-IXα levels in bile measured for each graft after 30 minute reperfusion. As shown in FIG. 2(B), the ZnPP-induced reduction of bilirubin fluxes in the grafts, i.e., the difference in bilirubin-IXα levels between the hemin-treated cold-preserved grafts reperfused in the presence of ZnPP (Hemin/C16+ZnPP) and in the absence of ZnPP (Hemin/C16) is greater than that between the hemin-treated grafts (Hemin/C16) and the hemin-untreated grafts (C16).

Moreover, the hemin-untreated grafts receiving 16-hour cold preservation and subsequent 30 minute reperfusion (C16) secreted bilirubin at about 200 pmol/min/g liver, which is comparable to the level of bilirubin secretion measured for grafts receiving 30 minute control perfusion without cold preservation (see Document 28). When the grafts were treated with hemin, cold-preserved for 16 hours and then reperfused for 30 minutes, bilirubin fluxes were significantly increased and about 1.5-fold higher than those of the hemin-untreated group. However, this increase is considerably lower than the 3-fold increase in venous CO fluxes observed for the same group (FIG. 1(B)). Such stoichiometric inconsistency between these two heme degradation products may arise from reduced efficiency of bilirubin transport in the hemin-treated grafts receiving 16-hour cold preservation. When compared to co-perfusion with CuPP instead of ZnPP, co-perfusion with ZnPP caused a significant decrease in bilirubin levels. This suggested that the inhibitory effect of ZnPP against bilirubin secretion would rely on its inhibitory effect against HO activity.

### Example 4

### Bilirubin-Induced Recovery of Graft Dysfunction

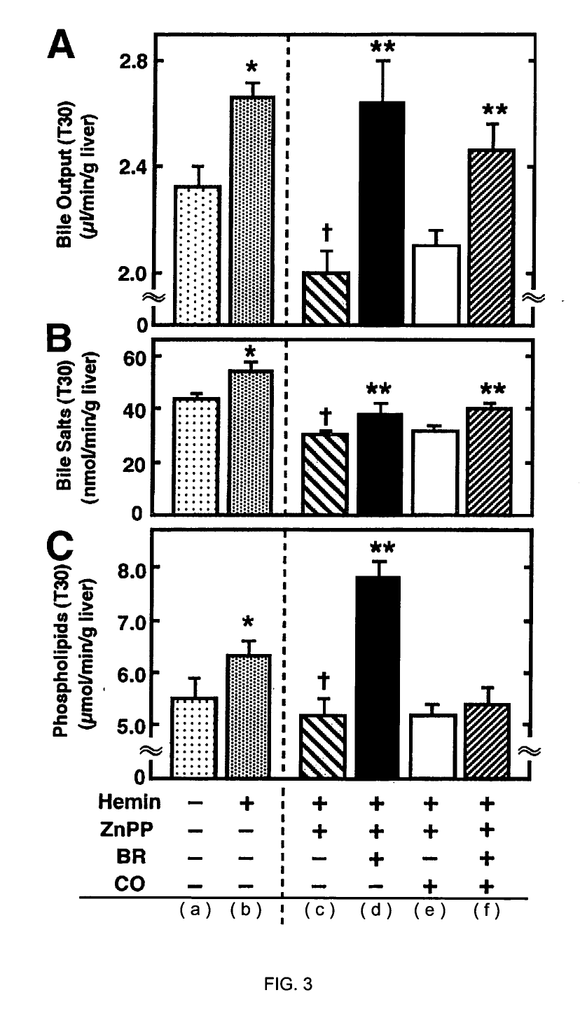

To examine whether bilirubin and/or CO could recover the HO-1-inducing effect inhibited by ZnPP, a strong HO activity inhibitor, the following experiment was carried out. The grafts were reperfused with carbogen-saturated Krebs-Henseleit buffer (pH 7.4, 37° C.) supplemented with bilirubin (Sigma Chemical, Co.) and/or CO in combination with 1 μmol/L ZnPP. In relation to the concentration of bilirubin, based on the data showing that the plasma bilirubin concentration in portal blood samples collected from hemin-treated rats was 3.2±0.8 μmol/L (n=4) and the plasma bilirubin concentration in control rats was 1.0 μmol/L or less (see Document 29), the physiologically relevant concentration of bilirubin in hemin-treated livers was estimated to be about 5 μmol/L. In the presence of ZnPP, the grafts receiving 16-hour cold preservation were co-perfused for 30 minutes with the following: (c) no bilirubin and no CO, (d) 5 μmol/L bilirubin, (e) 5 μmol/L CO, or (f) 5 μmol/L bilirubin and 5 μmol/L CO, followed by measuring the levels of (A) bile output, (B) bile salts in bile, and (C) phospholipids in bile secreted from the grafts. Similarly, hemin-untreated (a) and hemin-treated (b) grafts were provided in the absence of ZnPP for use as controls. The total amounts of bile salts and phospholipids in bile samples were measured by enzyme inhibition assay according to a reported method (see Document 23). The results obtained are shown in FIGS. 3(A), (B) and (C).

As shown in FIG. 3(A), the hemin-treated grafts (b) showed an increase in bile output when compared to the hemin-untreated grafts (a), but co-perfusion with ZnPP (c) canceled the effect of hemin treatment and reduced bile output below the level given by perfusion in the absence of ZnPP (a). This ZnPP-induced reduction of bile output was recovered by co-perfusion with bilirubin (d). This effect of bilirubin could not achieved by co-perfusion with CO at the same concentration (e). Moreover, co-perfusion with bilirubin and CO (f) failed to enhance the effect given by co-perfusion with bilirubin alone (d).

### Example 5

### Bilirubin Doses and Graft Dysfunction Recovery

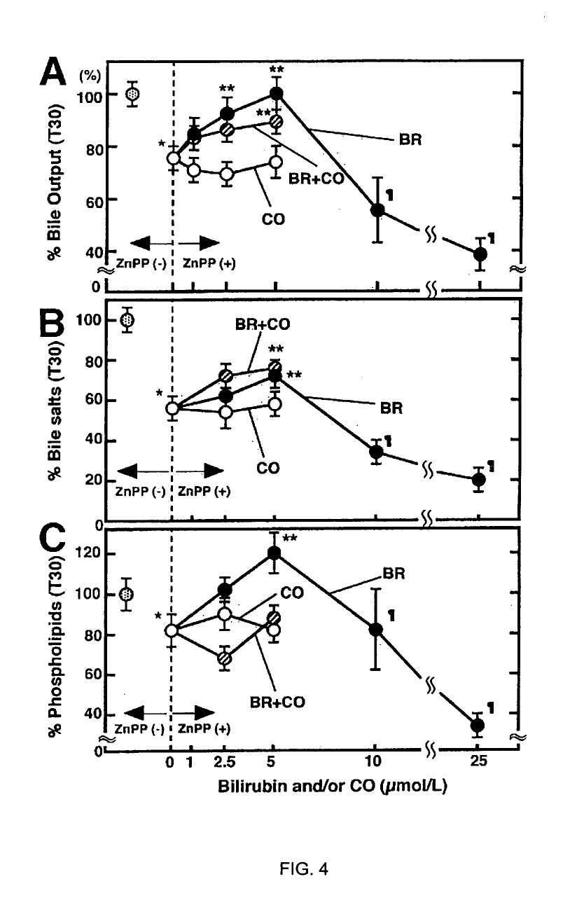

An attempt was made to examine dose-dependent effects of bilirubin and/or CO during co-perfusion. The same experiment as described in Example 4 was repeated, except that the doses of bilirubin and/or CO were varied. After being cold-preserved for 16 hours, the hemin-treated grafts were reperfused for 30 minutes in the presence of ZnPP using reperfusate containing various concentrations of bilirubin and/or CO, and then measured for the levels of bile output, bile salts and phospholipids in bile from the grafts, which were determined in percentages relative to those obtained by reperfusion in the absence of ZnPP. The results obtained are shown in FIGS. 4(A), (B) and (C).

As shown in FIG. 4, in the grafts receiving 16-hour cold preservation and subsequent 30 minute reperfusion, the percentages of bile output, bile salts and phospholipids in bile were significantly recovered at a bilirubin concentration of 5 μmol/L or less in the reperfusate, but were reduced at a concentration of 5 μmol/L or more. This indicated that the effect of bilirubin on liver grafts would change at a concentration around 5 μmol/L in the perfusate. In contrast, 30 minute reperfusion at a CO concentration of 5 μmol/L or less resulted in no recovery of either bile output from the grafts or bile salt and phospholipid levels in bile. These results indicated that bilirubin made more contributions than CO to the recovery of bile functions in the hemin-treated liver grafts.

### Example 6

### Recovery of Liver Cell Damage by Bilirubin Administration

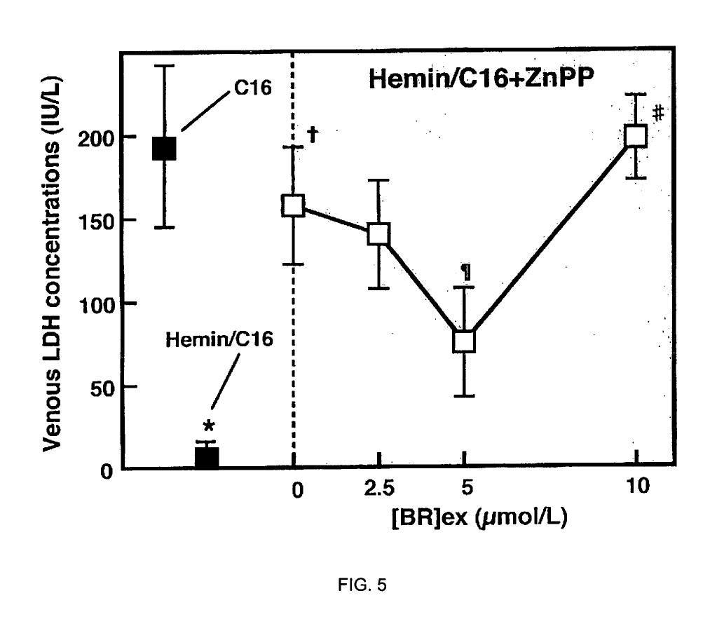

As in the case of Example 5, after being cold-preserved for 16 hours, the hemin-treated grafts were reperfused for 30 minutes in the presence of ZnPP using reperfusate containing varying concentrations of bilirubin, and then measured for the amount of lactate dehydrogenase (LDH) released into the hepatic venous perfusate, which was indicative of liver cell damage in the grafts. For use as controls, hemin-untreated grafts receiving 16-hour cold preservation (C16) and hemin-treated grafts receiving 16-hour cold preservation (Hemin/C16) were provided in the absence of ZnPP. The amount of LDH was measured as described in an earlier document (see Document 24). The results obtained are shown in FIG. 5.

As shown in FIG. 5, in the hemin-untreated control (C16), 16-hour cold preservation caused a significant increase in the amount of venous LDH release, which was indicative of reduced cell survival. The hemin-treated control (Hemin/C16) showed almost complete inhibition of LDH release. During reperfusion, LDH release was significantly increased upon co-perfusion with ZnPP, but the amount of LDH release decreased until the bilirubin concentration in the reperfusate reached 5 μmol/L. The amount of LDH release reached a minimum at a bilirubin concentration of 5 μmol/L and then increased with increase in bilirubin concentration. This means that the ZnPP-induced inhibition of hemin treatment effects was recovered depending on the concentration of bilirubin supplemented exogenously at up to 5 μmol/L. However, the risk of cell damage was not improved by 10 μmol/L bilirubin administration under the conditions of this experiment. These results indicated that the reduction of cell survival would depend on the action of HO and that bilirubin played an important role in the HO-1-mediated protection mechanism for post-cold ischemic liver grafts.

### Example 7

### Ex Vivo Bilirubin Co-Perfusion Time and Reperfusion Injury

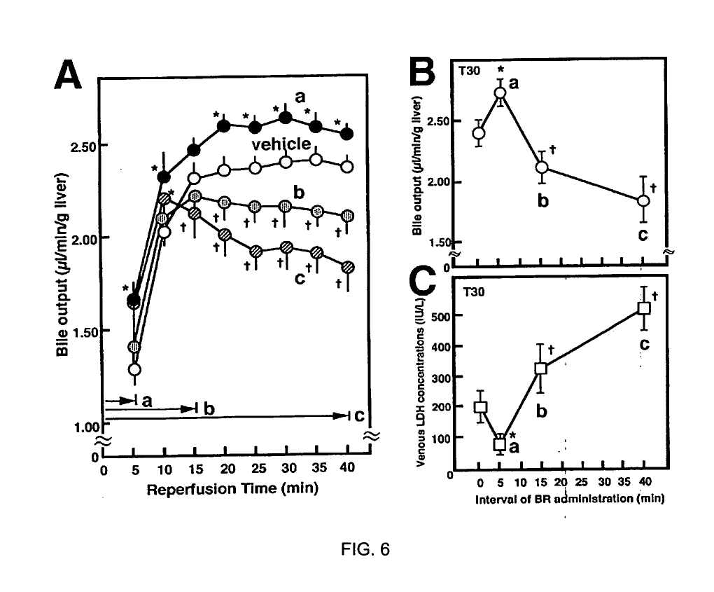

Since Example 6 suggested that bilirubin played an important role in the HO-1-mediated graft protection, further attempts were made to examine whether acute disturbances of ex vivo bile secretion and cell survival recovery could be prevented by simply adding bilirubin (bile pigment) to reperfusate without performing hemin treatment on the grafts. Since bilirubin has been found to cause decreases in bile output and cell survival when administered at high concentrations, the concentration of bilirubin was set to 5 μmol/L. Bilirubin was added to carbogen-saturated Krebs-Henseleit buffer (pH 7.4, 37° C.) to give a concentration of 5 μmol/L and the grafts cold-preserved for 16 hours were reperfused by one-way perfusion with this buffer in such a manner that the buffer was supplied from the portal veins to liver cells in the grafts. The grafts were divided into the following test groups: (a) receiving 5 minute reperfusion with the bilirubin-containing buffer and subsequent 35 minute reperfusion in the absence of bilirubin, (b) receiving 15 minute reperfusion with the bilirubin-containing buffer and subsequent 25 minute reperfusion in the absence of bilirubin, and (c) receiving 40 minute perfusion with the bilirubin-containing buffer. As a control group (d), the cold-preserved grafts received only bilirubin-free perfusion for 40 minutes. Bile output was then measured over time for each group. The results obtained are shown in FIG. 6(A). Further, FIGS. 6(B) and (C) show the relationship between reperfusion time with the bilirubin-containing buffer and bile output from the grafts and the relationship between reperfusion time and venous LDH concentration, respectively.

As shown in FIG. 6(A), the grafts receiving 5 minute bilirubin reperfusion (a) showed a significant improvement in bile output when compared to the bilirubin-free control (d). The grafts receiving prolonged reperfusion with the bilirubin-containing buffer for 15 minutes (b) and 40 minutes (c) showed a significant decrease in bile output when compared to the control (d) and the test group (a) receiving 5 minute reperfusion with the bilirubin-containing buffer. Moreover, increases in LDH release, indicative of graft damage, were observed concurrently with bilirubin dose-dependent bile output into the hepatic vein. This indicated that prolonged reperfusion with the bilirubin-containing buffer would cause cell damage.

As discussed above in Examples 4 to 6, when the hemin-treated grafts were cold-preserved for 16 hours and reperfused for 30 minutes with 5 μmol/L bilirubin in the presence of ZnPP, there was no adverse effect on bile secretion or cell survival in the hemin-treated liver grafts. This means that the competence to exogenous bilirubin differs between hemin-treated and hemin-untreated grafts. Taking into account the observation from Examples 2 and 3 indicating that there was inconsistency between venous CO fluxes and bile bilirubin secretion in the hemin-treated grafts, these results suggest that pharmaceutical graft pretreatment for HO-1 induction would modify physiological processes of bilirubin entry and secretion.

[In Vivo Experiments]

### Example 8

### Liver Transplantation Model

To examine in vivo effects of HO-1 preconditioning, a model experiment for liver isograft transplantation was carried out. Male inbred Lewis rats of 220-280 g were allowed to drink water without any restriction, fasted for 24 hours before the experiment, and provided for use as donors and recipients for liver transplantation. In HO-1 preconditioning groups, the rats were hemin-treated at 18 hours before graft preparation in the same manner as used in the ex vivo experiment of Example 1. Liver transplantation was performed according to the technique developed by Kamada (see Document 25) with minor modifications. More specifically, the donors were anesthetized by intraperitoneal injection of pentobarbital sodium (40 mg/kg body weight) and then laparotomized. Intraperitoneal organs including the liver were immediately flushed via the ventral aorta with 20 mL lactated Ringer's solution of 4° C. and then washed with 10 mL UW solution kept at 4° C. Subsequently, 10 ml cold UW solution was slowly injected via the portal vein into each liver. A PE-10 catheter for bile sample collection was inserted into the common bile duct of each liver. The livers were isolated from their surrounding tissues and preserved in UW solution at 4° C. for 16 hours.

In the recipient operation, the UW solution remaining in the above liver grafts preserved for 16 hours was rinsed off using various irrigation solutions, followed by isograft transplantation in the following manner. The graft hepatic vein was sutured and anastomosed to the recipient's hepatic superior vena cava using 7-0 Prolene. The portal vein and hepatic inferior vena cava were anastomosed using cuff techniques. The hepatic vein ligature time was set to constant in the range of 12 to 14 minutes for all experimental groups. The hepatic artery was not reconstructed. The bile duct was not anastomosed and an intubation tube was led out of the body for bile sample collection after the operation. The recipients surviving for 24 hours were sacrificed under ether anesthesia to collect their blood samples.

### Example 9

### Protective Effects of Bilirubin Against Post-Transplantation Cholestasis and Injury

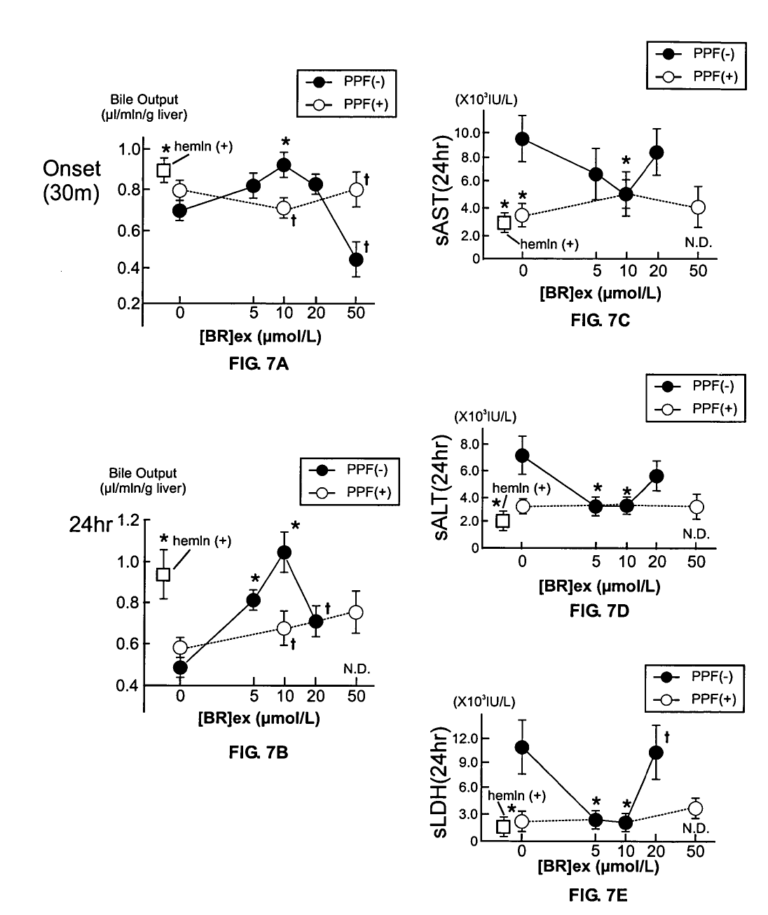

In light of Example 7 showing that short-term washing with bilirubin was effective for recovery of post-reperfusion stasis and cell injury in the liver grafts cold-preserved for 16 hours, further attempts were made to examine the in vivo effect of bilirubin washing against post-transplantation injury in the cold-preserved grafts. At the end of the preservation period, lactated Ringer's solution (4° C.) or bilirubin-containing Ringer's solution containing various concentrations of bilirubin in lactated Ringer's solution (4° C., treatment agent for organ graft rinse) was used to effect washing/rinsing prior to transplantation. Washing/rinsing was accomplished by closed circulation in which the lactated Ringer's solution or bilirubin-containing Ringer's solution was injected via the portal veins into the liver grafts, followed by recipient operation as described above. The level of bile output was measured at 30 minutes and 24 hours after initiation of reperfusion. The results obtained are shown in FIGS. 7(A) and (B) (PPF(−)). To determine the degree of graft injury at 24 hours after initiation of reperfusion, blood levels of aspartate aminotransferase (AST), alanine aminotransferase (ALT) and LDH were measured in a standard manner. The results obtained are shown in FIGS. 7(C), (D) and (E) (PPF(−)).

As shown in FIGS. 7(A) and (B), when the grafts were washed/rinsed by perfusion via the portal veins with free bilirubin-containing Ringer's solution prior to transplantation, there was a difference in bile output levels between 30 minutes and 24 hours after initiation of reperfusion. When the grafts were washed with 5 to 10 μmol/L bilirubin-containing Ringer's solution, bile output was significantly improved to the same level as hemin treatment, whereas cholestasis occurred when using Ringer's solution containing a higher concentration of bilirubin. None of the rats receiving grafts washed with 50 μmol/L bilirubin-containing Ringer's solution were able to survive for 24 hours after transplantation (N.D. in FIG. 7(B)). The levels of s(serum)AST, sALT and sLDH at 24 hours after transplantation in the grafts rinsed with bilirubin-containing irrigation solution also varied depending on the content of bilirubin. As shown in FIGS. 7(C), (D) and (E), graft washing at a bilirubin concentration of 5 to 10 μmol/L allowed reduction of cell injury to the same level as hemin treatment, whereas washing with irrigation solution containing a higher concentration of bilirubin caused exacerbation of cell injury. As indicated by the in vivo degree of post-reperfusion bile stasis and cell injury upon graft washing at a bilirubin concentration of 5 to 10 μmol/L, washing with low concentration bilirubin could replace hemin treatment in ameliorating post-transplantation reperfusion injury.

### Example 10

### Confirmation of the In Vivo Effects of Bilirubin in Graft Protection

Next, in relation to the effectiveness and accessibility of bilirubin in graft protection, further attempts were made to examine changes in the effect and accessibility of bilirubin in the presence of plasma proteins capable of capturing free bilirubin. Several grafts were treated with irrigation solution containing a human plasma protein fraction (PPF, Baxter Healthcare Corporation), which was commonly used in clinical transplantation (see Document 26). PPF is primarily composed of albumin (96% by weight or more), and other components are globulin and electrolytes. The concentration of albumin in the irrigation solution was adjusted to about 4.4 g/dl. Since bilirubin can bind to albumin at an equimolar ratio, the concentration of free bilirubin in the PPF-containing irrigation solution will be almost zero even when the solution contains bilirubin. Such a bilirubin-containing irrigation solution supplemented with PPF was used for reperfusion in the same manner as used in Example 9, except that closed circulation was replaced by open circulation, followed by measuring the levels of bile output at 30 minutes and 24 hours after initiation of reperfusion. The results obtained are shown in FIGS. 7(A) and (B) (PPF(+)). The levels of sAST, sALT and sLDH were also measured at 24 hours after initiation of reperfusion. The results obtained are shown in FIGS. 7(C), (D) and (E) (PPF(+)).

As shown in FIGS. 7(A) to (E), when the grafts were washed/rinsed by open circulation with 10 μmol/L bilirubin-containing irrigation solution in the presence of PPF, such washing/rinsing did not contribute to the increases in bile output at 30 minutes and 24 hours after initiation of reperfusion, but offset the adverse effects produced by 50 μmol/L bilirubin-containing irrigation solution. On the other hand, the addition of PPF to the irrigation solution significantly reduced the degree of cell injury. This effect was comparable to the case where the grafts were washed with 10 μmol/L bilirubin alone. These results confirmed that bilirubin was responsible for the increase in bile output and the reduction in the degree of cell injury observed when using the bilirubin-containing irrigation solution. In addition, although PPF can be present around cells and prevent cell death (0 μmol/L bilirubin in PPF(+) of FIGS. 7(C) to (E)), it cannot penetrate into cells and hence has no effect in improving bile secretory functions (0 μmol/L bilirubin in PPF(+) of FIG. 7(B)). In contrast, bilirubin is a lipophilic small-molecular antioxidant and is transported into cells, thus believing that bilirubin reduces the degree of cell injury and improves bile secretory functions. It is therefore suggested that bilirubin having penetrated into cells prevents cell dysfunction (abnormal bile secretion) caused by reactive oxygen which is converted from oxygen contained in the rinse solution upon switching cold preservation to warm washing.

### Example 11

### Accessibility of Bilirubin to Graft Liver Cells

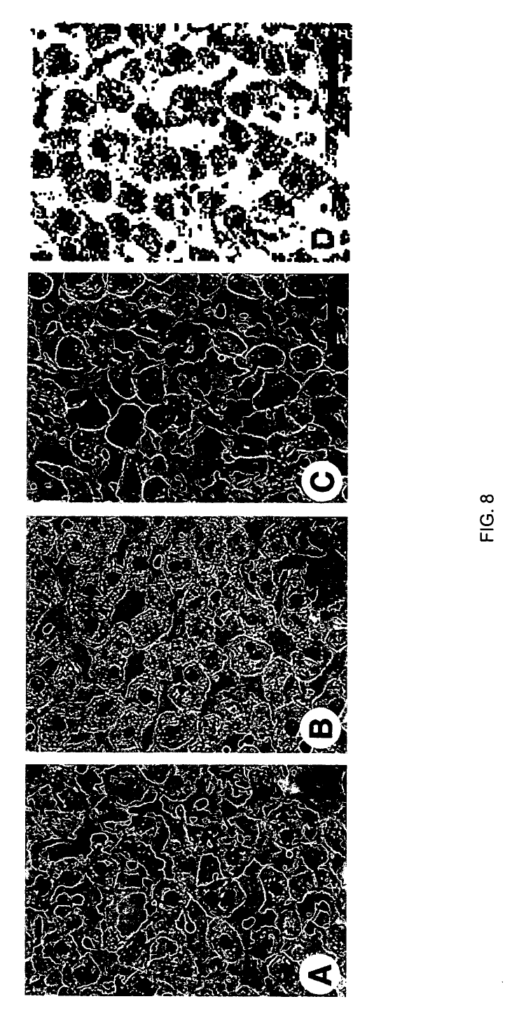

To examine the effect of bilirubin washing on the accessibility of bilirubin to graft tissues, immunohistological detection of bilirubin was performed using the anti-bilirubin-IXα monoclonal antibody 24G7 according to a previously reported method of the inventors (see Document 27). The results obtained are shown in FIG. 8. As observed in the grafts perfused with bilirubin-free lactated Ringer's solution (A), the monoclonal antibody 24G7 triggered an immune reaction primarily in liver non-parenchymal cells including Kupffer cells. This immune reaction was also detected slightly in parenchymal cell regions when compared to negative control (B), suggesting the presence of endogenous bilirubin as a major cellular component derived by HO-1. When the grafts were washed with bilirubin-containing Ringer's solution, bilirubin was highly absorbed into liver parenchymal cells to induce a significant enhancement of the immune reaction (C). In contrast, when the grafts were washed with bilirubin-containing Ringer's solution supplemented with PPF, the accessibility of bilirubin was canceled by co-perfusion with PPF and the immune reaction in liver parenchymal cells was reduced to the level given by washing with bilirubin-free lactated Ringer's solution (D). These results indicate that graft washing with bilirubin-containing Ringer's solution allows bilirubin to effectively reach graft liver cells.

According to the present invention, when organ grafts are perfused with an antioxidant-containing solution, which is to be incorporated into cells, to wash the grafts before being transplanted, the solution can penetrate into cells in the grafts with high efficiency to eliminate reactive oxygen, thus enabling prevention of post-transplantation reperfusion injury (e.g., graft dysfunction) and also enabling safer and simpler transplantation. Moreover, the present invention also achieves organ transplantation at low cost and allows prevention of reperfusion injury even in transplantation after cold ischemic preservation, thus making it possible to expand the range of organ transplantation.

The following journal documents or other documents including patent gazettes cited herein are incorporated by reference throughout the specification.
1. Clavien P A, Harvey P R C, Strasberg S M. Transplantation 1992; 53:957-978.
2. Lemasters J J, Bunzendahl H, Thurman R G Liver Transpl Surg 1995; 1:124-138.
3. Greig P D, Woolf G M, Sinclair S B, Abecassis M, Strasberg S M, Taylor B R, Blendis L M, Superina R A, Glynn M F X, Langer B, Levy G A. El. Transplantation 1989; 48:447-453.
4. Kumamoto Y, Suematsu M, Shimazu M, Kato Y, Sano T, Makino N, Hirano K, Naito M, Wakabayashi C, Ishimura Y, Kitajima M. Hepatology 1999; 30:1454-1463.
5. Nakamura T, Arii S, Monden K, Sasaoki T, Adachi Y, Ishiguro S, Fujita S, Mizumoto M, Imamura M. J Surg Res 1996; 62:207-215.
6. Kukan M, Vajdova K, Horecky J, Nagyova A, Mehendale H M, Trnovec T. Hepatology 1997; 26:1250-1257.
7. Takahashi Y, Tamaki T, Tanaka M, Konoeda Y, Kawamura A, Katori M, Kakita A. Transplant Proc 1998; 30:3700-3702.
8. Matsumoto K, Honda K, Kobayashi N. Protective Transplantation 2001; 71:862-868.
9. Yin D P, Sankary H N, Chong A S F, Ma L L, Shen J, Foster P, Williams J W. Transplantation 1998; 66:152-157.
10. Amersi F, Buelow R, Kato H, Ke B, Coito A J, Shen X D, Zhao D, Zaky J, Melineck J, Lassman C R, Kolls J K, Alam J, Ritter T, Volk H D, Farmer D G, Ghobrial R M, Busuttil R W, Kupiec-Weglinski J W. J Clin Invest 1999; 104:1631-1639.
11. Soares M P, LinY, Anrather J, Csizmadia E, Takigami K, Sato K, Grey S T, Colvin R B, Choi A M, Poss K D, Bach F H. Nature Med 1998; 4:1073-1077.
12. Maines M D. FASEB J 1988; 2:2557-2568.
13. Maines M D, Trakshel G M, Kutty R K. J Biol Chem 1986; 261:411-419.
14. Ponka P, Beaumont C, Richardson D R. J Biol Chem 1998; 35:35-54.
15. Poss K D, Tonegawa S. Proc Natl Acad Sci USA 1997; 94:10919-10924.
16. Suematsu M, Goda N, Sano T, Kashiwagi S, Egawa T, Shinoda Y, Ishimura Y. J Clin Invest 1995; 96:2431-2437.
17. Wakabayashi Y, Takamiya R, Mizuki A, Kyokane T, Goda N, Yamaguchi T, Takeoka S, Tsuchida E, Suematsu M, Ishimura Y. Am J Physiol Gastrointest Liver Physiol 1999; 277:G 1088-G1096.
18. Suematsu M, Ishimura Y Hepatology 2000; 31:3-6.
19. Stocker R, Yamamoto Y, McDonagh A F, Glazer A N, Ames B N. Science 1987; 235:1043-1046.
20. Goda N, Suzuki K, Naito M, Takeoka S, Tsuchida E, Ishimura Y, Tamatani T, Suematsu M. J Clin Invest 1998; 101:604-612.
21. Shimizu S, Izumi Y, Yamazaki M, Shimizu K, Yamaguchi T, Nakajima H. Biochim Biophys Acta 1988; 967:255-260.
22. Izumi Y, Yamazaki M, Shimizu S, Shimizu K, Yamaguchi T, Nakajima H. Biochim Biophys Acta 1988; 967:261-266.
23. Tanaka A, Katagiri K, Hoshino M, Hayakawa T, Tsukada K, Takeuchi T. Am J Physiol Gastrointest Liver Physiol 1994; 266:G324-G329.
24. Morikawa N, Suematsu M, Kyokane T, Goda N, Kumamoto Y, Okitsu T, Ishimura Y, Kitajima M. Hepatology 1998; 28:1289-1299.
25. Kamada N, Calne R Y. Transplantation 1979; 28:47-50.
26. Todo S, Nery J, Yanaga K, Podesta L, Gordon R D, Starzl T E. JAMA 1989; 261:711-714.
27. Ozawa N, Goda N, Makino N, Yamaguchi T, Yoshimura Y, Suematsu M. J Clin Invest 2002; 109:457-467.
28. Yamaguchi T, Wakabayashi Y, Tanaka M, Sano T, Ishikawa H, Nakajima H, Suematsu M, Ishimura Y. Am J Physiol Gastrointest Liver Physiol 1996; 270:G1028-G1032.
29. Hayashi S, Takamiya R, Yamaguchi T, Matsumoto K, Tojo S J, Tamatani T, Kitajima M, Makino N, Ishimura Y, Suematsu M. Circ Res 1999; 85:663-671.
30. International Publication No. WO98/13055

1. A method of treating an organ for transplantation, which comprises perfusing an organ graft with an organ preservation solution, preserving the organ graft under cold conditions for a given period of time, and reperfusing the organ graft with an antioxidant-containing solution, which is to be incorporated into cells, to wash the organ graft before being transplanted.

2. The method of treating an organ for transplantation according to claim 1, wherein the antioxidant-containing solution to be incorporated into cells is a bilirubin-containing solution.

3. The method of treating an organ for transplantation according to claim 2, wherein the bilirubin-containing solution is a 5 to 10 μmol/L bilirubin-containing solution.

4. The method of treating an organ for transplantation according to claim 2, wherein the bilirubin-containing solution is reperfused for 3 to 10 minutes to wash the organ graft.

5. The method of treating an organ for transplantation according to claim 1, wherein the organ is liver, heart, kidney, pancreas or lung.

6. A treatment agent for organ graft rinse used in reperfusing an organ graft, which has been perfused with an organ preservation solution and then cold-preserved for a given period of time, to wash the organ graft before being transplanted, wherein said treatment agent for organ graft rinse comprises, as an active ingredient, an antioxidant-containing solution to be incorporated into cells.

7. The treatment agent for organ graft rinse according to claim 6, wherein the antioxidant-containing solution to be incorporated into cells is a bilirubin-containing solution.

8. The treatment agent for organ graft rinse according to claim 7, wherein the bilirubin-containing solution is a 5 to 10 μmol/L bilirubin-containing solution.

9. The treatment agent for organ graft rinse according to claim 6, wherein the organ is liver, heart, kidney, pancreas or lung.

10. A method of organ transplantation, which comprises perfusing an organ graft with an organ preservation solution, preserving the organ graft under cold conditions for a given period of time, reperfusing the organ graft with an antioxidant-containing solution, which is to be incorporated into cells, to wash the organ graft before being transplanted, and transplanting the organ graft.

11. The method of organ transplantation according to claim 10, wherein the organ transplantation is isograft transplantation.

12. The method of organ transplantation according to claim 10, wherein the antioxidant-containing solution to be incorporated into cells is a bilirubin-containing solution.

13. The method of organ transplantation according to claim 12, wherein the bilirubin-containing solution is a 5 to 10 μmol/L bilirubin-containing solution.

14. The method of organ transplantation according to claim 12, wherein the bilirubin-containing solution is reperfused for 3 to 10 minutes to wash the organ graft.

15. The method of organ transplantation according to claim 10, wherein the organ is liver, heart, kidney, pancreas or lung.

16. A method of organ preservation, which comprises perfusing an organ with an organ preservation solution, preserving the organ under cold conditions for a given period of time, and reperfusing the organ with an antioxidant-containing solution, which is to be incorporated into cells, to wash the organ before being used.

17. The method of organ preservation according to claim 16, wherein the antioxidant-containing solution to be incorporated into cells is a bilirubin-containing solution.

18. The method of organ preservation according to claim 17, wherein the bilirubin-containing solution is a 5 to 10 μmol/L bilirubin-containing solution.

19. The method of organ preservation according to claim 17, wherein the bilirubin-containing solution is reperfused for 3 to 10 minutes to wash the organ.

20. The method of organ preservation according to claim 16, wherein the organ is liver, heart, kidney, pancreas or lung.
